(19) United States
(12) Patent Application Publication
(10) Pub. No.: US 2015/0377747 A1
(43) Pub. Date: Dec. 31, 2015

(54) A SAMPLE HANDLING DEVICE

(71) Applicant: Sirma CAPAR DINCER, Cankaya Ankara (TR)

(72) Inventor: Sirma CAPAR DINCER, Ankara (TR)

(21) Appl. No.: 14/766,758

(22) PCT Filed: Feb. 10, 2014
(86) PCT No.: PCT/TR2014/000029

§ 371 Date: Aug. 10, 2015

(30) Foreign Application Priority Data
Feb. 11, 2013 (TR) 2013/01591

(51) Int. Cl. G01N 1/20; C12Q 1/24

(52) U.S. Cl. CPC G01N 1/20; C12Q 1/24

(57) ABSTRACT

Sample handling device which enables to take samples (samples of living beings) from single celled invertebrate living beings which live in different depths of aqueous mediums such as lakes, ponds, barrages and sea shore zones. The aim is to take samples from vertebrate living beings which live by attaching themselves on living beings or non living materials such as plants, stones and shells in aqueous medium such as lakes, ponds, barrages and shore zones. Another aim is to present the medium where single cell living beings live and to carry such living beings to the laboratory in order to examine them.

### TECHNICAL FIELD

The invention is related to a sample handling device which enables to take samples (samples of living beings) from single celled invertebrate living beings which live in different depths of aqueous mediums such as lakes, ponds, barrages and sea shore zones.

### BACKGROUND

The depths of aqueous mediums such as lakes, ponds and barrages are not equal to each other at each location. The physical and chemical characteristics of water differ depending on changing depths and/or other environmental factors, thus each living water creature prefers to live at a depth suitable for itself. In order to be able to carry out a certain determination regarding a certain living group living in an aqueous medium, samples from all different depths possible must be taken. However, it is difficult for such a process to be carried out in lakes which have a wide water area and depth. In order to solve this problem, samples are taken from reference depths that have been predetermined.

In order to catch the invertebrate living beings living in aqueous mediums to take samples; equipment such as, Van Dorn sampler at horizontal and vertical direction and plankton scoopers, ekman scooper, sediment sieves, sediment core samplers are used. However these equipments only give good results when used on macro vertebrate living beings, zooplanktonic and phytoplanktonic living beings. The 10 micrometer pore dimension plankton scooper gives good results when sampling gastrotricha single cell (ciliophora) examined within the protozoa phylum.

In the Chinese beneficial model application numbered CN2662643 a double sampling product with vertical dimensions in order to examine sea sources has been mentioned. With said device, examination by taking samples from small sea creatures primarily planktons are carried out.

The plankton scooper is a great tool to take samples within a water column, however it is not suitable to be used at shore zones. For this reason at shore zones, in order to take samples from living beings which attach themselves onto other living beings or non living materials such as plants, stones and shells may not be possible in many living being groups. In such areas, equipment which are fixed and which can be colonized and be attached onto the living being and which can stay put such as the living and non living materials are needed.

### SUMMARY OF THE INVENTION

The present invention aims to take samples from invertebrate living beings which live by attaching themselves on living beings or non living materials such as plants, stones and shells in aqueous medium such as lakes, ponds, barrages and shore zones. Another aim of the present invention is, to present the medium where single cell living beings live and to carry such living beings to the laboratory in order to examine them.

### BRIEF DESCRIPTION OF THE DRAWINGS

The sample handling device developed in order to reach the aims of the invention has been illustrated in the attached figures, wherein said figures illustrate the following:

FIG. 1: General view of the sample handling device subject to the invention

FIG. 2 General view of the microscope slide section of the sample handling device subject to the invention.

FIG. 3 General view of the microscope slide slot section of the sample handling device subject to the invention

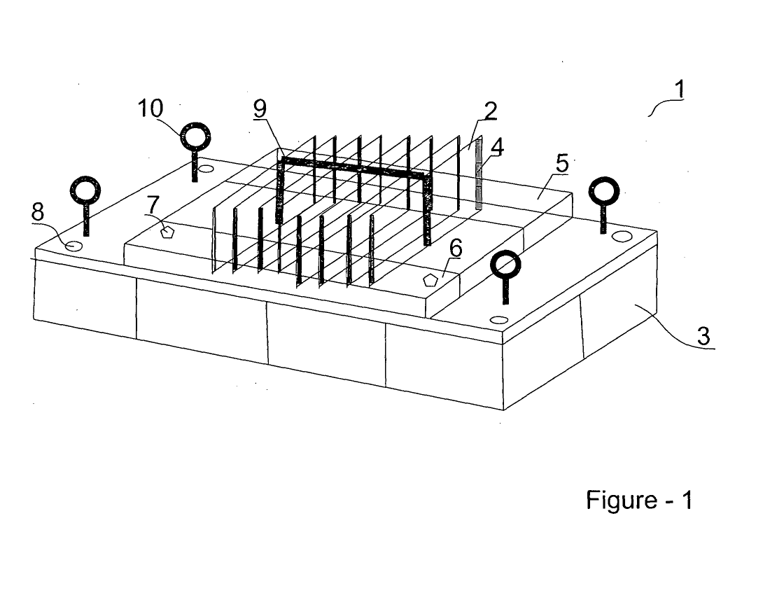

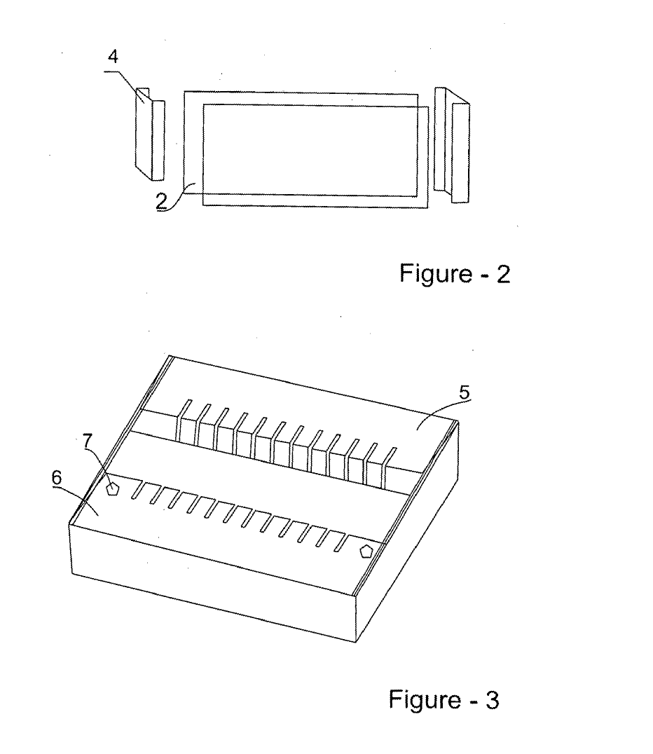

### DETAIL DESCRIPTION OF THE INVENTION

The parts in the figures have each been numbered and their references have been listed below.

1. Sample handling device

2. Microscope slide

3. Weight chamber

4. Plastic frame

5. Fixed microscope slide slot

6. Mobile microscope slide slot

7. Lock

8. Fixer

9. Supporting member

10. Hook

The sample handling device (1) subject to the invention basically comprises a microscope slide (2) used for collecting samples and sampling, a fixed microscope slide (5) where the slides (2) are placed in, and a mobile slide slot (6), a plastic frame (4) used to connect two slides (2), a lock (7) used to fix the mobile slide slot (6), a supporting member (9) used to prevent the breakage of slides (2) and to ensure they stay fixed, and a hook (10).

Said sample handling device (1), is left in different depths of aqueous mediums such as lakes, ponds, barrages, and sea shore zones for a sufficient amount of time to establish a new living area. In order to represent the exact medium where single celled living beings live, by attaching themselves to solid materials, the sample handling device (1) and slides (2) are produced from transparent, light and durable materials such as PVC, or rigid glass etc.

At least 10 slides (2) are used in order to take the sufficient amount of samples from the living beings that attach themselves onto the sample handling device (1) and the number of slides (2) on the device (1) can be increased depending on the amount of samples. The slides (2) are placed in to fixed and mobile slide slots (5 and 6) in two groups by the aid of plastic frames (4) in order for one face of said slides (2) to stay clean.

The guide rails where the slides (2) have been placed in the fixed and mobile slide slot (5 and 6), need to be opened at equal width. In the case where the rail widths are different from each other the slides (2) may come out of their slots due to water flow. The slides (2) that are placed inside the guide rails within the fixed slide slot (5) can be stabilized by using a mobile slide slot (6). The mobile slide slot (6) that is used to ensure that the slides (2) do not come out of the fixed slide slot (5) are fixed to the sample handling device (1) by means of locks (7).

At least a space of 3 cm is needed between the sample handling device (1) and the fixed and mobile slide slot (5 and 6) where the slides (2) are placed. Said space enables the flow of water and allows the equal distribution of living creatures that attach onto the slide (2).

In order for the sample handling device (1) to submerge into water easily, a cavity can be opened at its bottom as desired. Together with this, a weight chamber (3) is used so that the sample handling device (1) can stay fixed during the preferred amount of time, at the desired depth, without it being negatively affected from external factors such as inclination, flow etc. Additional balancing weights determined in accordance to the depth of the water can be added into the partitions within the weight chamber (3) which is mounted to the sample handling device (1) by means of fixers (8).

Ropes are used to pull back the sample handling device (1) which has been kept under water for a certain amount of time, to the surface of the water. Said ropes are tied to at least 2 preferably 4 hooks (10) that have been fixed/mounted to the corners of the bottom base of the sample handling device (1).

It is thus possible to carry the living creatures that have attached themselves on the microscopic slides (2) from their habitats, without said samples being damaged, by placing the device (1) which has been submerged into different depths of water, into a water filled container and as a result it is possible to examine said organisms while they are still living.

1. A sample handling device configured to take samples from single celled invertebrate living beings which live in different depths of aqueous mediums, wherein the aqueous mediums is selected from a group consisting of lakes, ponds, barrages and sea shore zones, comprising:
a microscope slide produced from transparent, light and sustainable material selected from a group consisting of PVC, rigid glass, etc, used for collecting samples and sampling;
a fixed microscope slide slot containing the guiderails where the microscope slides are placed on and, wherein the fixed microscope slide slot has at least a 3 cm space between the sample handling device;
a mobile microscope slide slot having at least a 3 cm space between the sample handling device, wherein the mobile microscope slide slot is used to stabilize the microscope slides placed on the guide rails located in the fixed microscope slide slot;
a lock used to fix the mobile microscope slide slot onto the sample handling device;
a supporter used to ensure that the slides do not move and to prevent them from breaking.
at least two hooks fixed/mounted to the corner sides of the bottom base of the sample handling device in order to ensure that the sample handling device is pulled back out onto the water surface;
a plastic frame connecting two microscope slides such that one face of the two microscope slides stay clean.

2. The sample handling device according to claim 1, further comprising:
a weight chamber mounted to the sample handling device so that the sample handling device stays fixed during the desired period of time without being affected from external factors of inclination or flow at desired water depths, and the weight chamber has partitions in which additional balancing weights capable of be placed in accordance with the depth of the water;
a fixer enabling to attach the weight chamber onto the sample handling device.

3. The sample handling device according to claim 2, further comprising a cavity opened at its bottom so that the sample handling device submerges into water easily.

4. The sample handling device according to claim 1, further comprising a space of at least 3 cm between the sample handling device and the fixed and mobile slots, enabling the flow of water and allowing the equal distribution of living creatures.

5. The handling device according to claim 1, wherein the at least two hooks comprise four hooks.
